(19) United States
(12) Patent Application Publication
(10) Pub. No.: US 2025/0275750 A1
(43) Pub. Date: Sep. 4, 2025

(54) COUPLANT COATING MECHANISM AND ULTRASONIC IMAGING SYSTEM

(71) Applicant: GE Precision Healthcare LLC, Waukesha, WI (US)

(72) Inventors: Lu Jin, Wuxi (CN); Qiang Yao, Wuxi (CN); Liping Chen, Wuxi (CN); Hongyu Zhao, Wuxi (CN)

(21) Appl. No.: 19/070,217

(22) Filed: Mar. 4, 2025

(30) Foreign Application Priority Data
Mar. 4, 2024 (CN) 202410244489.0

(51) Int. Cl. A61B 8/00

(52) U.S. Cl. CPC A61B 8/4281

(57) ABSTRACT

A couplant coating mechanism including an engagement portion engaging with a scanning probe, the engagement portion being provided with a first channel; and an accommodation portion connected to the engagement portion, the accommodation portion including a main body portion for accommodating a couplant, an internal space of the main body portion being in communication with the first channel, and the couplant accommodated in the main body portion flowing out along the first channel.

### CROSS-REFERENCE TO RELATED APPLICATION

This application claim priority to Chinese Patent Application No. 202410244489.0, which was file on Mar. 4, 2024 at the Chinese Patent Office. The entire contents of the above-listed application are incorporated by reference herein in their entirety.

### TECHNICAL FIELD

The present application relates to the field of medical imaging, and in particular to a couplant coating mechanism and an ultrasonic imaging system.

### BACKGROUND

Ultrasonic imaging is one of the important means for imaging the interior of the body of a person to be scanned. Generally, ultrasonic imaging systems use ultrasonic transducers to convert electrical energy into ultrasonic pulses. The ultrasonic pulses are sent to the interior of the body of the person to be scanned, and then echo signals are generated. The echo signals are received by a transducer element and then converted into electrical signals. The electrical signals are processed by a specialized processing device to then form a desired ultrasonic image.

Ultrasonic imaging systems have important applications in scanning many organs of the body. For example, a full-field breast ultrasonic scanning device may be used to image breast tissue in one or more planes. During a full-field breast ultrasonic scanning process, it is generally necessary to apply a couplant to the body of a patient prior to scanning.

It should be noted that the above introduction of the background is only for the convenience of clearly and completely describing the technical solutions of the present application, and for the convenience of understanding for those skilled in the art. The above technical solutions are not considered to be well known to those skilled in the art merely because they are set forth in the Background of the present application.

### SUMMARY

The inventors have found that, at present, a method for applying a couplant to a patient comprises a technician squeezing the couplant from a bottle to apply the couplant to the body of the patient, and then using a tool to apply the couplant slowly and uniformly to the body of the patient. This process occupies the total time of scanning and reduces scanning efficiency, and there is a problem that uneven application caused by manual operation affects the scanning effect.

In view of at least one of the above problems or other similar problems, embodiments of the present application provide a couplant coating mechanism and an ultrasonic imaging system. Automatic application of a couplant is implemented by adding the couplant coating mechanism to a scanning probe, thereby saving scanning time, avoiding couplant waste, and further improving scanning efficiency and user experience.

According to one aspect of the embodiments of the present application, provided is a couplant coating mechanism. The couplant coating mechanism comprises:

An engagement portion engaging with a scanning probe, the engagement portion being provided with a first channel; and

An accommodation portion connected to the engagement portion, the accommodation portion comprising a main body portion for accommodating a couplant, an internal space of the main body portion being in communication with the first channel, and the couplant accommodated in the main body portion flowing out along the first channel.

In some embodiments, the accommodation portion further comprises an extension portion connected to the main body portion, the extension portion is provided with a second channel that is in communication with the internal space of the main body portion, and the internal space of the main body portion is in communication with the first channel via the second channel.

In some embodiments, there are two extension portions oppositely disposed, and each extension portion is provided with the second channel.

In some embodiments, the engagement portion comprises two wall portions, and a connection portion connecting the two wall portions; and a gap between the two wall portions is used for accommodating the scanning probe, and each wall portion is provided with the first channel.

In some embodiments, there are a plurality of first channels disposed extending from top to bottom.

In some embodiments, extending ends of the two wall portions each have a couplant outlet in communication with the first channel.

In some embodiments, the couplant outlet is an integral structure formed along the extending end, and the curvature of the couplant outlet matches that of an extending end of the scanning probe.

In some embodiments, the height of the couplant outlet is higher than the height of an extending end of the scanning probe.

In some embodiments, the accommodation portion further comprises:

An extruding portion, the extruding portion being disposed inside the main body portion and capable of moving to extrude the couplant; and

A driving portion, the driving portion driving the extruding portion to move.

In some embodiments, the pressing portion comprises a first flipping plate and a second flipping plate that divide the internal space of the main body portion into two parts, and the driving portion comprises a first motor and a second motor; and the first motor is capable of driving the first flipping plate to move, so that the couplant on a side of the first flipping plate flows out, and the second motor is capable of driving the second flipping plate to move, so that the couplant on a side of the second flipping plate flows out.

In some embodiments, the main body portion is provided with two openings, the two openings being in communication with two internal spaces, respectively, which are separated by the first flipping plate and the second flipping plate.

In some embodiments, the main body portion further comprises:

An electrical connection portion, the electrical connection portion being connected to the driving portion to transmit electrical energy to the driving portion,

The electrical connection portion comprising at least one of:

An electrical receiving terminal, the electrical receiving terminal being disposed on an inner side of the main body portion or the engagement portion and cooperating with a power supply terminal of the scanning probe to receive the electrical energy; and

A cable, the cable receiving electrical energy from outside the couplant coating mechanism.

According to another aspect of the embodiments of the present application, provided is an ultrasonic imaging system. The system comprises:

A scanning assembly, comprising a scanning probe; and

The couplant coating mechanism according to any one of the foregoing embodiments.

In some embodiments, the scanning assembly further comprises a frame, the frame accommodating the scanning probe and a driving device, the driving device driving the scanning probe to move within the frame to perform ultrasonic scanning.

One advantageous effect of the embodiments of the present application is that automatic application of a couplant is implemented by adding the couplant coating mechanism to the scanning probe, thereby saving scanning time, avoiding couplant waste, and further improving scanning efficiency and user experience.

With reference to the following description and drawings, specific implementations of the present application are disclosed in detail. It should be understood that the implementations of the present application are not limited in scope thereby. Within the scope of the spirit and clauses of the appended claims, the embodiments of the present application include many changes, modifications, and equivalents.

The features described and/or illustrated for one implementation may be used in one or more other implementations in the same or similar manner, be combined with features in other embodiments, or replace features in other implementations.

It should be emphasized that the term “include/comprise/have”, when used herein, refers to the presence of features, integrated components, or assemblies, but does not preclude the presence or addition of one or more other features, integrated components, or assemblies.

### BRIEF DESCRIPTION OF THE DRAWINGS

The above and other objects, features and advantages of the embodiments of the present application will become more apparent from the following detailed description with reference to the drawings, in which:

FIG. 1 is a perspective view of an ultrasonic imaging system according to embodiments of the present application;

FIG. 2 is a block diagram of the ultrasonic imaging system according to embodiments of the present application;

FIG. 3 is a schematic diagram of a couplant coating mechanism according to embodiments of the present application;

FIG. 4 is a schematic diagram of an installation position of a couplant coating mechanism according to embodiments of the present application;

FIG. 5 is a schematic diagram of a couplant coating mechanism viewed from another angle;

FIG. 6 is a schematic exploded view of a couplant coating mechanism according to embodiments of the present application;

FIG. 7 is a schematic diagram of a partial structure of an accommodation portion of a couplant coating mechanism according to embodiments of the present application;

FIG. 8 is another schematic diagram of a couplant coating mechanism according to embodiments of the present application; and

FIG. 9 is still another schematic diagram of a couplant coating mechanism according to embodiments of the present application.

### DETAILED DESCRIPTION

The foregoing and other features of the embodiments of the present application will become apparent from the following description with reference to the drawings. In the description and drawings, specific implementations of the present application are disclosed in detail, and part of the implementations in which the principles of the present application may be employed are indicated. It should be understood that the present application is not limited to the described implementations, and include all modifications, variations, and equivalents which fall within the scope of the appended claims.

In the embodiments of the present application, the terms “first”, “second”, “upper”, “lower”, etc. are used to distinguish different elements with respect to naming, but do not represent a spatial arrangement, a temporal order, etc. of these elements, and these elements should not be limited by these terms. The term “and/or” includes any and all combinations of one or more associated listed terms. The terms “comprise”, “include”, “have”, etc., refer to the presence of described features, elements, components, or assemblies, but do not exclude the presence or addition of one or more other features, elements, components, or assemblies.

In the embodiments of the present application, the singular forms “a” and “the”, etc., include plural forms, and should be broadly construed as “a type of” or “a class of” rather than being limited to the meaning of “one”. Furthermore, the term “the” should be construed as including both the singular and plural forms, unless otherwise specified in the context. In addition, the term “according to” should be construed as “at least in part according to . . . ” and the term “on the basis of” should be construed as “at least in part on the basis of . . . ”, unless otherwise specified in the context.

The features described and/or illustrated for one embodiment may be used in one or more other embodiments in an identical or similar manner, combined with features in other embodiments, or replace features in other embodiments.

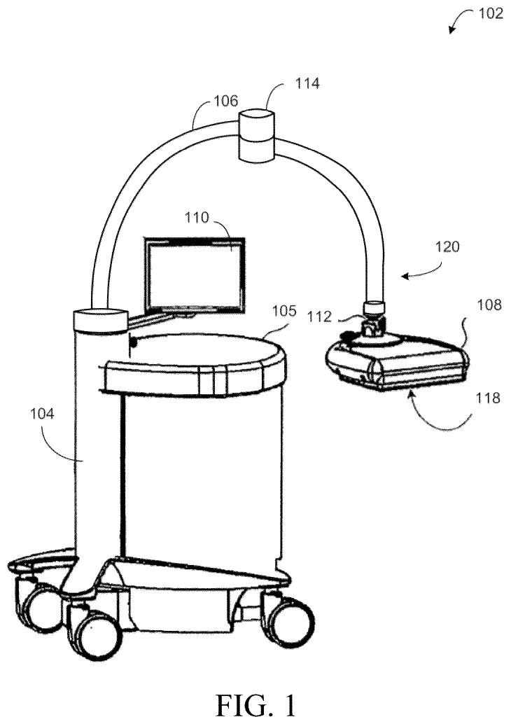

FIG. 1 shows a perspective view of an ultrasonic imaging system 102 according to some embodiments. As shown in FIG. 1, the body of the ultrasonic imaging system 102 may include a main device, a display 110, an adjustable arm 106, and a scanning assembly 108. The main device may include a body frame 104, an ultrasonic processor housing 105, and an ultrasonic processor inside the housing 105. The specific structure of each component will be illustrated in detail below.

The body frame 104, the ultrasonic processor housing 105 containing the ultrasonic processor, a movable and adjustable support arm (for example, an adjustable arm) 106 including a hinge joint 114, the scanning assembly 108 connected to a first end 120 of the adjustable arm 106 by means of a ball and socket connector (for example, a ball joint) 112, and the display 110 connected to the body frame 104. The display 110 is connected to the body frame 104 at a joining point where the adjustable arm 106 enters the body frame 104. Since the display 110 is directly connected to the body frame 104 rather than the adjustable arm 106, the display 110 does not affect the weight of the adjustable arm 106 and a balancing mechanism of the adjustable arm 106. In one example, the display 110 is rotatable in horizontal and transverse directions (for example, rotatable around a central axis of the body frame 104), but is not vertically movable. In an alternative example, the display 110 may also be vertically movable. Although FIG. 1 depicts the display 110 connected to the body frame 104, in other examples, the display 110 may be connected to different components of the imaging system 102, such as, connected to the ultrasonic processor housing 105, or positioned away from the imaging system 102.

In one embodiment, the adjustable arm 106 is configured and adapted such that the pressing/scanning assembly 108 (i) is neutrally buoyant in space, or (ii) has a light net downward weight (for example, 1-2 kg) for pressing the breast, while allowing easy user operation. In an alternative embodiment, the adjustable arm 106 is configured such that the scanning assembly 108 is neutrally buoyant in space during positioning of a scanner on tissue of a patient. Then, after the scanning assembly 108 is positioned, internal components of the imaging system 102 may be adjusted to apply a desired downward weight for pressing the breast and improving image quality. In one example, the downward weight (for example, a force) may be in the range of 2-11 kg.

As described above, the adjustable arm 106 includes the hinge joint 114. The hinge joint 114 divides the adjustable arm 106 into a first arm portion and a second arm portion. The first arm portion is connected to the scanning assembly 108 and the second arm portion is connected to the body frame 104. The hinge joint 114 allows the second arm portion to rotate relative to the second arm portion and the body frame 104. For example, the hinge joint 114 allows the scanning assembly 108 to translate transversely and horizontally, but not vertically, relative to the second arm portion and the body frame 104. In such manner, the scanning assembly 108 can rotate toward the body frame 104 or away from the body frame 104. However, the hinge joint 114 is configured to allow the entire adjustable arm 106 (for example, the first arm portion and the second arm portion) to move vertically together as a whole (for example, translating upward and downward along with the body frame 104).

The scanning assembly 108 may include a film assembly 118 having a film that is in a substantially tensioned state to be at least partially attached, for pressing the breast. The film assembly 118 has a bottom surface for contacting the breast, and when the bottom surface is in contact with the breast, the transducer sweeps over a top surface of the film to scan the breast. In one example, the film is a tensioned fabric sheet.

The film assembly 118 may further include an outer frame and a film. The film is fixedly disposed in the outer frame, and the outer frame is detachably connected to the scanning assembly. In an ultrasonic imaging process performed by the ultrasonic imaging system, one side surface of the film can be at least partially in contact with an ultrasonic transducer, and another side surface of the film is at least partially in contact with a tissue to be scanned. Such an arrangement can ensure that the ultrasonic transducer transmits and receives signals with less attenuation, and can fix the breast to be scanned to facilitate scanning.

Optionally, the adjustable arm may include a potentiometer (not shown) to allow position and direction sensing performed by the pressing/scanning assembly 108, or may use other types of position and direction sensing (such as gyroscope, magnetic, optical, and radio frequency (RF)). A fully functional ultrasonic engine may be provided within the ultrasonic processor housing 105, and is configured to drive the ultrasonic transducer, and generate volumetric breast ultrasound data from a scan in conjunction with related position and orientation information. In some examples, volumetric scan data may be transmitted to another computer system by using any of a variety of data transmission methods known in the art so as to be further processed, or the volumetric scan data may be processed by the ultrasonic engine. A general-purpose computer/processor integrated with the ultrasonic engine may further be provided for general user interface and system control. The general-purpose computer may be a self-contained stand-alone unit, or may be remotely controlled, configured, and/or monitored by remote stations connected across networks.

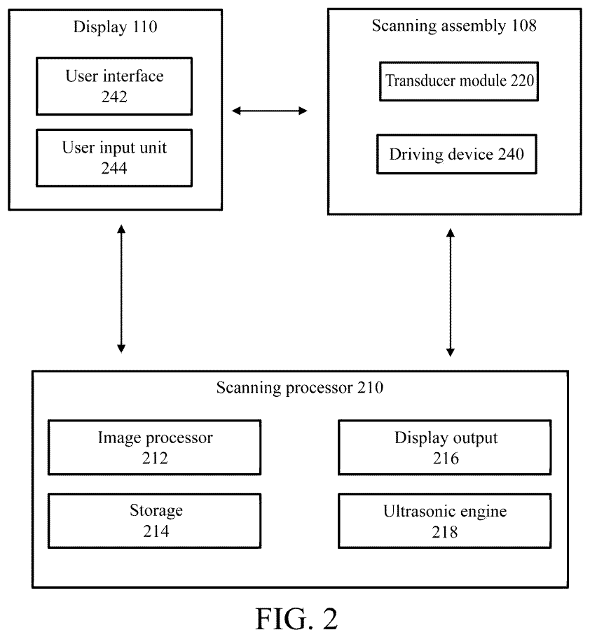

FIG. 2 is a block diagram 200 that schematically illustrates various system components of the ultrasonic imaging system 102. As shown in FIG. 2, the ultrasonic imaging system 102 includes a scanning assembly 108, a display 110, and a scanning processor 210. In one example, the scanning processor 210 may be included within an ultrasonic processor housing 105 of the imaging system 102. As shown in FIG. 2, the scanning assembly 108, the display 110, and the scanning processor 210 are separate components communicating with each other. However, in some embodiments, one or more of these components may be integrated (for example, the display and the scanning processor may be included in a single component).

In the example of FIG. 2, the scanning assembly 108 includes at least a transducer module 220 and a driving device 240. The transducer module 220 includes a transducer array of transducer elements, such as a piezoelectric element, which converts electrical energy into ultrasonic waves and then detects reflected ultrasonic waves.

The scanning assembly 108 may communicate with the scanning processor 210 to send raw scan data to an image processor. The scanning assembly 108 may optionally communicate with the display 110 so as to indicate a user to reposition the scanning assembly as described above, or to receive information from the user (via user input 244).

In the example of FIG. 2, the scanning processor 210 includes an image processor 212, a memory 214, display output 216, and an ultrasonic engine 218. The ultrasonic engine 218 may drive activation of the transducer elements of the transducer module 220, and in some embodiments, the driving device 240 may be activated. Furthermore, the ultrasonic engine 218 may receive raw image data (for example, ultrasonic echoes) from the scanning assembly 108. The raw image data may be sent to the image processor 212 and/or a remote processor (for example, via a network) and be processed to form a displayable image of a tissue sample. It should be understood that in some embodiments, the image processor 212 may be included in the ultrasonic engine 218.

In the example of FIG. 2, information may be transmitted from the ultrasonic engine 218 and/or the image processor 212 to the user of the imaging system 102 via the display output 216 of the scanning processor 210. In one example, the user of the ultrasonic imaging system may include an ultrasonic technician, a nurse, or a physician such as a radiologist. For example, a processed image of scanned tissue may be sent to the display 110 via the display output 216. In another example, information (such as the progress of scanning) related to parameters of the scanning may be sent to the display 110 via the display output 216. The display 110 may include a user interface 242 configured to display images or other information to the user. Furthermore, the user interface 242 may be configured to receive an input from the user (such as by means of a user input unit 244), and send the input to the scanning processor 210. In one example, the user input unit 244 may be a touch screen of the display 110. However, other types of user input mechanisms are also possible, such as a mouse, a keyboard, and the like.

The scanning processor 210 may further include the memory 214. The memory 214 may include movable and/or permanent devices, and may include an optical memory, a semiconductor memory, and/or a magnetic memory, etc. The memory 214 may include a volatile, non-volatile, dynamic, static, read/write, read only, random access, sequential access, and/or annex memory. The memory 214 may store non-transitory instructions executable by a controller or processor (such as a controller or the image processor 212) so as to perform one or more methods or routines. The memory 214 may store raw image data received from the scanning assembly 108, processed image data received from the image processor 212 or the remote processor, and/or additional information.

The embodiments of the present application provide a couplant coating mechanism.

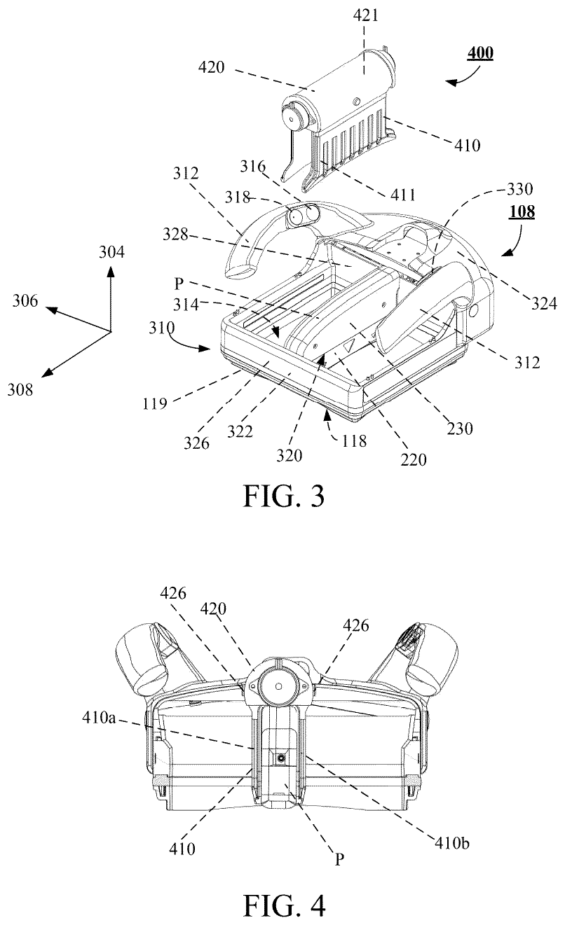

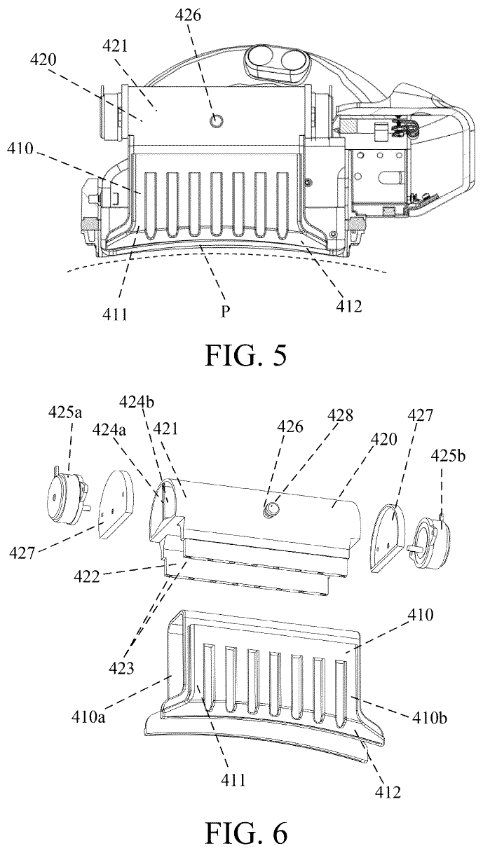

FIG. 3 is a schematic diagram of a couplant coating mechanism according to the embodiments of the present application, FIG. 4 is a schematic diagram of an installation position of the couplant coating mechanism according to the embodiments of the present application, and FIG. 5 is a schematic diagram of the couplant coating mechanism viewed from another angle.

As shown in FIG. 3 to FIG. 5, the couplant coating mechanism 400 according to the embodiments of the present application includes an engagement portion 410 engaging with a scanning probe P and an accommodation portion 420 connected to the engagement portion 410. The engagement portion 410 is provided with a first channel 411, the accommodation portion 420 includes a main body portion 421 for accommodating a couplant, an internal space of the main body portion 421 is in communication with the first channel 411, and the couplant accommodated in the main body portion 421 flows out along the first channel 411.

According to the foregoing embodiment, automatic application of a couplant is implemented by adding the couplant coating mechanism to the scanning probe, thereby saving scanning time, avoiding couplant waste, and further improving scanning efficiency and user experience.

In the foregoing embodiment, the engagement between the couplant coating mechanism and the scanning probe is not limited in manner, and may be implemented by means of an interference fit, or may be implemented by means of providing an engaging member, and any existing engagement manner is applicable to the above structure of the present application.

According to the above structure, an operator only needs to install the couplant coating mechanism on the scanning probe to implement automatic couplant coating, thereby improving the convenience of coating. In addition, since the couplant coating mechanism is engaged with the scanning probe, the couplant coating mechanism can be easily removed from the scanning probe when the couplant coating mechanism is not required, thereby improving convenience of use.

In some embodiments, as shown in FIG. 3, the scanning probe P is a part of a scanning assembly 108. The scanning assembly 108 includes a housing 310, a transducer module 220, and a module receiver 230. The transducer module 220 and the module receiver 230 constitute the foregoing scanning probe P.

The housing 310 includes a frame 322 and a handle portion 324, and the handle portion includes two handles 312. The two handles 312 oppose each other across a transverse axis of the scanning assembly 108, and the transverse axis is centered on the adjustable arm 106 and defined relative to a transverse axis 308. The frame 322 is rectangular, and an inner periphery of the frame 322 defines an opening 314. The opening 314 provides space (e.g., a void volume) for translating the module receiver 230 and the transducer module 220 during a scanning process. In another example, the frame 322 can have another shape, such as a square having the square opening 314. In addition, the frame 322 has a thickness defined between an inner periphery and an outer periphery of the frame 322.

The frame 322 includes four sets of side walls (e.g., a set including inner and outer side walls, the inner side walls defining the opening 314). In particular, the frame 322 includes a front side wall 326 and a rear side wall 328, the rear side wall 328 is directly connected to the handle portion 324 of the housing 310, and the front side wall 326 is opposite to the rear side wall 328 with respect to the horizontal axis 306. The frame 322 further includes right and left side walls, the corresponding side walls opposing each other and both being in a plane defined by the vertical axis 304 and the transverse axis 308.

The frame 322 of the housing 310 further includes a top side and a bottom side, and the top side and the bottom side are defined relative to the vertical axis 304. The top side faces the adjustable arm 106. The film 118 is disposed across the opening 314. More specifically, the film 118 is connected to the bottom side of the frame 322. In an example, the film 118 is a diaphragm that remains tensioned across the opening 314. The film 118 may be made from a flexible but non-stretchable material, and the material is thin, waterproof, durable, highly acoustically transparent, resistant to chemical corrosion, and/or biocompatible. As described above, the bottom surface of the film 118 may contact a tissue (e.g., a breast) during scanning, and the upper surface of the film 118 may at least partially contact the transducer module 220 during scanning. As shown in FIG. 3, the film 118 is permanently connected to a hard-housing holding portion 119 surrounding the periphery of the film 118. The holding portion 119 is connected to the bottom side of the frame 322. In one example, the holding portion 119 can be fastened to a lip-like edge on the bottom side of the frame 322 of the housing 310, so that the film 118 does not become unconnected during scanning, but is still removably connected to the frame 322. The film 118 may not be permanently connected to the hard-housing holding portion 119, and thus the film 118 may be connected to the frame 322 without the hard-housing holding portion 119. Instead, the film 118 may be directly and removably connected to the frame 322.

The handle portion 324 of the housing 310 includes the two handles 312 for moving the scanning assembly 108 in space and positioning the scanning assembly 108 on a tissue (e.g., on the body of a patient). In an alternative embodiment, the housing 310 may not include the handle 312. In an example, the handle 312 may be integrally formed with the frame 322 of the housing 310. In another example, the handle 312 and the frame 322 may be formed separately and then mechanically connected together to form the entire housing 310 of the scanning assembly 108.

In the example of FIG. 3, the scanning assembly 108 may be connected to the adjustable arm 106 by means of a ball joint 112 (e.g., a ball and socket connector). Specifically, a top dome portion of the handle portion 324 is connected to the ball joint 112. The top of the handle portion 324 includes a depression forming a socket, and a ball of the ball joint 112 is fit in the socket. The ball joint 112 is movable in multiple directions. For example, the ball joint 112 provides rotational motion of the scanning assembly relative to the adjustable arm 106. The ball joint 112 includes a locking mechanism for locking the ball joint 112 in place, thereby holding the scanning assembly 108 stationary relative to the adjustable arm 106. Furthermore, the ball joint 112 may also be configured to only rotate but not to move in multiple directions, such as oscillating.

Additionally, as shown in FIG. 3, the handle 312 of the handle portion 324 includes buttons for controlling scanning and adjusting the scanning assembly 108. Specifically, a first handle of the handles 312 includes a first weight adjustment button 316 and a second weight adjustment button 318. The first weight adjustment button 316 may reduce a load applied to the scanning assembly 108 from the adjustable arm 106. The second weight adjustment button 318 may increase a load applied to the scanning assembly 108 from the adjustable arm 106. Increasing the load applied to the scanning assembly 108 may increase the pressure and the amount of pressing applied to the tissue on which the scanning assembly 108 is placed. Furthermore, increasing the load applied to the scanning assembly increases the effective weight of the scanning assembly on the tissue to be scanned. In one example, increasing the load may press a tissue of a patient, such as a breast. In such manner, varying amounts of pressure (e.g., load) may be applied consistently with the scanning assembly 108 during scanning, so as to obtain high quality images by using the transducer module 220.

Before the scanning process, a user (e.g., an ultrasonic technician or physician) may position the scanning assembly 108 on a patient or a tissue. Once the scanning assembly 108 is properly positioned, the user may adjust a weight (e.g., adjusting an amount of pressing) of the scanning assembly 108 on the patient by using the first weight adjustment button 316 and/or the second weight adjustment button 318. Then, the user may initiate the scanning process by means of additional control on the handle portion 324 of the housing 310. For example, as shown in FIG. 3, the second handle of the handles 312 includes two additional buttons 330 (not separately shown). The two additional buttons 330 may include a first button for initiating a scan (e.g., once the scanning assembly has been placed on the tissue/patient and an amount of pressing has been selected) and a second button for stopping the scan. In one example, once the first button is selected, the ball joint 112 may be locked, thereby stopping transverse and horizontal movement of the scanning assembly 108.

The module receiver 230 is positioned within the housing 310. Specifically, the module receiver 230 is mechanically connected to a first end of the housing 310 at a rear side wall 328 of the frame 322, and the first end is closer to the adjustable arm 106 than a second end of the housing 310. The second end of the housing 310 is located at a front side wall 326 of the frame 322. In one example, the module receiver 230 is connected to the first end via a protruding portion of the module receiver 230, and the protruding portion is connected to a motor of the module receiver 230.

As described above, the housing 310 is configured to remain stationary during scanning. In other words, once the weight applied to the scanning assembly 108 is adjusted by means of the adjustable arm 106 and then the ball joint 112 is locked, the housing 310 may remain in the resting position without translating in the horizontal or transverse direction. However, the housing 310 may still translate vertically as the adjustable arm 106 move vertically.

Instead, the module receiver 230 is configured to translate relative to the housing 310 during scanning. As shown in FIG. 3, the module receiver 230 translates horizontally along a horizontal axis 306 relative to the housing 310. The motor of the module receiver 230 may slide the module receiver 230 along an upper surface of the first end of the housing 310.

The transducer module 220 is removably connected to the module receiver 230. Therefore, during scanning, the transducer module 220 and the module receiver 230 translate horizontally. During scanning, the transducer module 220 sweeps horizontally across the breast under the control of the motor of the module receiver 230, and at the same time, a contact surface of the transducer module 220 contacts the film 118. The transducer module 220 and the module receiver 230 are connected together at a module interface 320. The module receiver 230 has a width that is the same as that of the transducer module 220. In an alternative embodiment, the width of the module receiver may be different from that of the transducer module 220. In some embodiments, the module interface 320 includes a connector between the transducer module 220 and the module receiver 230, and the connector includes mechanical and electrical connections.

The foregoing provides an illustrative description of the structure of the scanning assembly 108, but the present application is not limited thereto. The couplant coating mechanism according to the embodiments of the present application may alternatively be engaged with a scanning probe of a scanning assembly having another structure.

FIG. 6 is a schematic exploded view of a couplant coating mechanism according to embodiments of the present application.

As shown in FIG. 6, in some embodiments, an accommodation portion 420 further includes an extension portion 422 connected to a main body portion 421, the extension portion 422 is provided with a second channel 423 that is in communication with an internal space of the main body portion 421, and the internal space of the main body portion 421 is in communication with the first channel 411 via the second channel 423. Thus, a couplant accommodated in the main body portion 421 can flow into the first channel 411 along the second channel 423, and flow out.

In some embodiments, as shown in FIG. 6, there are two extension portions 422 oppositely disposed, and each extension portion 422 is provided with the second channel 423 described above. Thus, the couplant accommodated in the main body portion 421 of the accommodation portion 420 can flow out from both sides in a moving direction of the scanning probe P, thereby improving uniformity and stability of couplant coating.

In some embodiments, as shown in FIG. 6, an engagement portion 410 has two wall portions 410a and 410b, a gap between the two wall portions 410a and 410b is used for accommodating the scanning probe P, that is, after the couplant coating mechanism is assembled to the scanning probe P, the two wall portions 410a and 410b are located on both sides in the moving direction of the scanning probe P, respectively, as shown in FIG. 4. In FIG. 4, the left-right direction is the moving direction of the scanning probe P.

In some embodiments, as shown in FIG. 6, there are a plurality of first channels 411 disposed extending from top to bottom; eight first channels are shown in FIG. 6. By means of providing a plurality of first channels 411, uniformity of extrusion of the couplant can be ensured and usage can be reduced as much as possible.

In the foregoing embodiments, the quantity of the second channels 423 disposed on each extension portion 422 is the same as the quantity of the first channels 411 disposed on the wall portion of a corresponding side. As shown in FIG. 6, the quantity of the second channels 423 is the same as the quantity of the first channels 411, and eight of both being provided. The quantities of the second channels 423 and the first channels 411 disposed on the extension portions 422 and the wall portions on different sides may be the same or different. For example, there are eight second channels 423 and first channels 411 on one side, and there are seven second channels 423 and first channels 411 on the other side, which may be specifically configured according to requirements.

In the foregoing embodiment, as shown in FIG. 6, the width of the first channel 411 may be larger than the width of the second channel 423, and the projection of the second channel 423 in the up-down direction falls within the range of the first channel 411. Thus, when the couplant flows out from the main body portion 421 of the accommodation portion 420, the couplant may flow out first along the second channel 423 having a smaller width and then flow out along the first channel 411 having a larger width, thereby ensuring smooth flowing out of the couplant and avoiding clogging during the flowing of the couplant.

The foregoing is just an example, and the present application is not limited thereto. The first channel 411 and the second channel 423 may also have the same width and completely penetrate through one another, which can also ensure smooth flowing of the couplant and avoid clogging.

In the foregoing embodiment, the connection manner between the extension portion 422 and the engagement portion 410 is not limited as long as it is ensured that the second channel 423 and the first channel 411 can penetrate through, and the couplant can enter the first channel 411 from the second channel 423 and flow out from a couplant outlet 412 along the first channel 411.

In some embodiments, as shown in FIG. 6, extending ends of the two wall portions 410a and 410b each have a couplant outlet 412 that is in communication with the first channel 411. The through-type couplant outlet 412 can ensure that a small amount of couplant can be uniformly dispersed after passing through the first channel 411, thereby achieving a uniform coating effect even in a case of a small amount of couplant.

In the foregoing embodiments, as shown in FIG. 6, the couplant outlet 412 may be an integral structure formed along the extending ends of the foregoing wall portions 410a and 410b, that is, the couplant outlet 412 penetrates through, and the couplant flowing out from any first channel 411 flows into the couplant outlet 412, and is coated on a skin surface of a patient from the couplant outlet 412. For example, when the scanning probe P moves in one direction (for example, the right side in FIG. 4), the couplant flows out from the couplant outlet 412 on the right side in FIG. 4. When the scanning probe P moves in the other direction (for example, the left side in FIG. 4), the couplant flows out from the couplant outlet 412 on the left side in FIG. 4.

In some embodiments, as shown in FIG. 5, the curvature of the couplant outlet 412 matches that of an extending end of the scanning probe P. Thus, it can be ensured that the couplant outlet 412 does not affect the scanning of the patient during a scanning process.

In some embodiments, as shown in FIG. 5, the height of the couplant outlet 412 is higher than that of the extending end of the scanning probe P. Thus, it can be further ensured that the couplant outlet 412 does not affect the scanning of the patient during a scanning process.

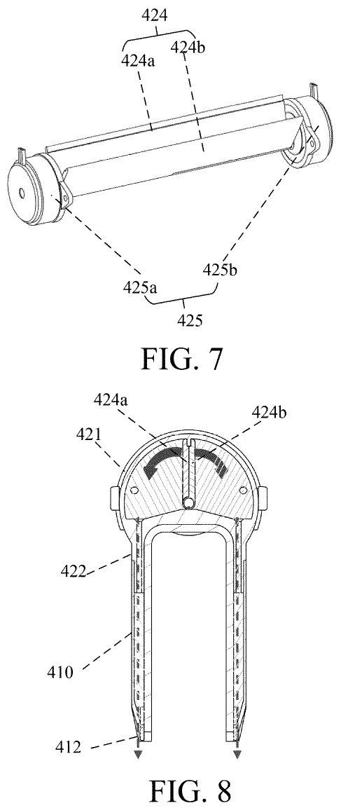

FIG. 7 is a schematic diagram of a partial structure of an accommodation portion 420 of a couplant coating mechanism according to embodiments of the present application.

As shown in FIG. 6 and FIG. 7, in some embodiments, the accommodation portion 420 further includes an extruding portion 424 disposed inside the main body portion 421 and a driving portion 425 that drives the extruding portion 424 to move. The extruding portion 424 moves under the driving of the driving portion 425 to extrude the couplant inside the main body portion 421. Thus, the couplant accommodated in the main body portion 421 can flow into the couplant outlet 412 along the second channel 423 and the first channel 411, thereby implementing automatic coating of the couplant.

In some embodiments, as shown in FIG. 6 and FIG. 7, the extruding portion 424 includes a first flipping plate 424a and a second flipping plate 424b that divide the internal space of the main body portion 421 into two parts, and the driving portion 425 includes a first motor 425a and a second motor 425b. The first motor 425a is capable of driving the first flipping plate 424a to move, so that the couplant on a side of the first flipping plate 424a flows out; and the second motor 425b is capable of driving the second flipping plate 424b to move, so that the couplant on a side of the second flipping plate 424b flows out.

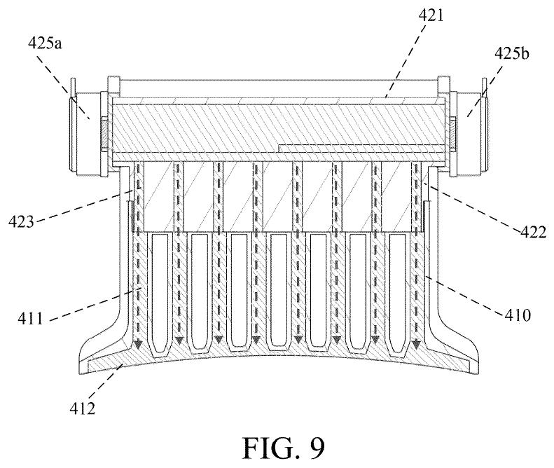

FIG. 8 is another schematic diagram of a couplant coating mechanism according to embodiments of the present application, showing a case in which the couplant coating mechanism is viewed from a side where a motor is located (for example, the left side shown in FIG. 6 or the right side shown in FIG. 6). FIG. 9 is yet another schematic diagram of a couplant coating mechanism according to embodiments of the present application, showing a case in which the couplant coating mechanism is viewed from a side where the wall portion 410a or 410b of the engagement portion 410 is located (for example, the left side shown in FIG. 4 or the right side shown in FIG. 4).

As shown in FIG. 8 and FIG. 9, when a scanning probe moves in a first direction, for example, when the scanning probe P moves to the left side shown in FIG. 4, a first motor 425a drives a first flipping plate 424a to rotate, and a couplant flows downward along a second channel 423 and a first channel 411 under the pressure of the first flipping plate 424a, then flows out from a couplant outlet 412, and is applied to a skin surface of a patient. Similarly, when the scanning probe moves in a second direction, for example, when the scanning probe P moves to the right side shown in FIG. 4, a second motor 425b drives a second flipping plate 424b to rotate, and the couplant flows downward along the second channel 423 and the first channel 411 under the pressure of the second flipping plate 424b, then flows out from the couplant outlet 412, and is applied to the skin surface of the patient. Thus, by means of the couplant coating mechanism, automatic application of a couplant is implemented, thereby saving scanning time, avoiding couplant waste, and further improving scanning efficiency and user experience.

According to the foregoing embodiment, in some possible implementations, the driving of the extruding portion 424 by the driving portion 425 is earlier than the movement of the scanning probe P. For example, as shown in FIG. 4, before the scanning probe P moves to the left side, the first motor 425a operates before the scanning probe P moves. After the couplant flows out from the couplant outlet 412 on the left side, the scanning probe P moves to the left side and starts operating. When the scanning probe P reaches the leftmost side, the first motor 425a stops operating, and the second motor 425b starts operating. After the couplant flows out from the couplant outlet 412 on the right side, the scanning probe P moves to the right side and starts operating. Thus, automatic coating of the couplant during a scanning process is implemented. The foregoing is just an example, and during a specific implementation process, the driving portion 425 and the scanning probe P may also operate at the same time, specifically depending on requirements.

In some embodiments, as shown in FIG. 4 to FIG. 6, the main body portion 421 is provided with two openings 426, and the two openings 426 are in communication with the two internal spaces divided by the first flipping plate 424a and the second flipping plate 424b, respectively, and are used by an operator to inject a couplant into the main body portion 421. Configured locations, sizes, and shapes of the openings 426 are not limited in the present application, depending on a specific requirement.

In some embodiments, the main body portion 421 further includes an electrical connection portion (not shown in the figures). The electrical connection portion is connected to the driving portion 425 to transmit electrical energy to the driving portion 425.

In the foregoing embodiments, a configured location of the electrical connection portion is not limited. In one possible implementation, the electrical connection portion includes an electrical receiving terminal. The electrical receiving terminal may be disposed on an inner side of the main body portion 421 or the engagement portion 410 and cooperates with a power supply terminal of the scanning probe P to receive the foregoing electrical energy. In another possible implementation, the electrical connection portion includes a cable. The cable receives electrical energy from outside the couplant coating mechanism.

The above embodiments merely provide illustrative descriptions of the embodiments of the present application. However, the present application is not limited thereto, and appropriate variations may be made on the basis of the above embodiments. For example, each of the above embodiments may be used independently, or one or more among the above embodiments may be combined.

In addition, the foregoing merely describes the structure of the couplant coating mechanism related to invention points of the present application. In a specific implementation, the couplant coating mechanism may further include other components used to implement other functions. As shown in FIG. 6, the accommodation portion 420 of the couplant coating mechanism may further include cover portions 427 between the motors 425a/425b and the main body portion 421 for covering both ends of the main body portion 421, a sealing member 428 for scaling the openings 426, and the like.

It can be learned from the foregoing embodiments that automatic application of a couplant is implemented by adding the couplant coating mechanism to the scanning probe, thereby saving scanning time, avoiding couplant waste, and further improving scanning efficiency and user experience. In addition, by means of the above structure, an operator only needs to install the couplant coating mechanism on the scanning probe to implement automatic coating of a couplant, thereby improving the convenience of coating. In addition, since the couplant coating mechanism is engaged with the scanning probe, the couplant coating mechanism can be easily removed from the scanning probe when the couplant coating mechanism is not required, thereby improving convenience of use.

The embodiments of the present application further provide an ultrasonic imaging system. The ultrasonic imaging system includes a scanning assembly and the couplant coating mechanism described in the foregoing embodiments. Related content of the couplant coating mechanism has been described above, which will not be described again here.

In the foregoing embodiments, the scanning assembly may further include a frame, for example, the frame 322 shown in FIG. 3. The frame 322 accommodates the described scanning probe (the transducer module 220 and the module receiver 230) and the driving device 240. The driving device 240 drives the scanning probe to move within the frame 322 to perform ultrasonic scanning. Since the composition and the operating manner of the scanning assembly are already described in the foregoing embodiments, details are not described again here.

In the embodiments of the present application, the combination of an automatically-driven scanning assembly and a couplant coating mechanism can improve scanning efficiency while improving user experience, especially as compared with a conventional hand-held ultrasonic probe. In one aspect, opposing upper surfaces of the scanning probe and the transducer in the scanning assembly provide sites of engagement for the couplant coating mechanism, whereas opposing sides of a transducer of a hand-held probe typically have cables or parts that need to be grasped by a physician, which makes it difficult to secure a couplant application mechanism. In another aspect, since the scanning probe in the scanning assembly is driven by the driving device, an additional couplant coating mechanism does not cause the physician to feel a lowering of user experience such as an increase in weight and an increase in volume, whereas it is difficult for a hand-held probe to achieve the described effect.

The present application is described above with reference to specific embodiments. However, it should be clear to those skilled in the art that the foregoing description is merely illustrative and is not intended to limit the scope of protection of the present application. Various variations and modifications may be made by those skilled in the art according to the spirit and principle of the present application, and these variations and modifications also fall within the scope of the present application.

Preferred embodiments of the present application are described above with reference to the accompanying drawings. Many features and advantages of the implementations are clear according to the detailed description, and therefore the appended claims are intended to cover all these features and advantages that fall within the true spirit and scope of these implementations. In addition, as many modifications and changes could be easily conceived of by those skilled in the art, the embodiments of the present application are not limited to the illustrated and described precise structures and operations, but can encompass all appropriate modifications, changes, and equivalents that fall within the scope of the implementations.

1. A couplant coating mechanism, comprising:
an engagement portion engaging with a scanning probe, the engagement portion being provided with a first channel; and
an accommodation portion connected to the engagement portion, the accommodation portion comprising a main body portion for accommodating a couplant, an internal space of the main body portion being in communication with the first channel, and the couplant accommodated in the main body portion flowing out along the first channel.

2. The couplant coating mechanism according to claim 1, wherein
the accommodation portion further comprises an extension portion connected to the main body portion, the extension portion is provided with a second channel that is in communication with the internal space of the main body portion, and the internal space of the main body portion is in communication with the first channel via the second channel.

3. The couplant coating mechanism according to claim 2, wherein
there are two extension portions oppositely disposed, and each extension portion is provided with the second channel.

4. The couplant coating mechanism according to claim 1, wherein
the engagement portion comprises two wall portions, and a connection portion connecting the two wall portions; and
a gap between the two wall portions is used for accommodating the scanning probe, and each wall portion is provided with the first channel.

5. The couplant coating mechanism according to claim 4, wherein
there are a plurality of first channels disposed extending from top to bottom.

6. The couplant coating mechanism according to claim 4, wherein
extending ends of the two wall portions each have a couplant outlet in communication with the first channel.

7. The couplant coating mechanism according to claim 6, wherein
the couplant outlet is an integral structure formed along the extending end, and the curvature of the couplant outlet matches that of an extending end of the scanning probe.

8. The couplant coating mechanism according to claim 6, wherein
the height of the couplant outlet is higher than the height of an extending end of the scanning probe.

9. The couplant coating mechanism according to claim 1, wherein the accommodation portion further comprises:
an extruding portion, the extruding portion being disposed inside the main body portion and being capable of moving to extrude the couplant; and
a driving portion, the driving portion driving the extruding portion to move.

10. The couplant coating mechanism according to claim 9, wherein
the extruding portion comprises a first flipping plate and a second flipping plate that divide the internal space of the main body portion into two parts, and the driving portion comprises a first motor and a second motor; and
the first motor is configured to drive the first flipping plate to move, so that the couplant on a side of the first flipping plate flows out, and
the second motor is configured to drive the second flipping plate to move, so that the couplant on a side of the second flipping plate flows out.

11. The couplant coating mechanism according to claim 10, wherein
the main body portion is provided with two openings, the two openings being in communication with two internal spaces, respectively, which are separated by the first flipping plate and the second flipping plate.

12. The couplant coating mechanism according to claim 9, wherein the main body portion further comprises:
an electrical connection portion, the electrical connection portion being connected to the driving portion to transmit electrical energy to the driving portion,
and the electrical connection portion comprising at least one of:
an electrical receiving terminal, the electrical receiving terminal being disposed on an inner side of the main body portion or the engagement portion, and cooperating with a power supply terminal of the scanning probe to receive the electrical energy; and
a cable, the cable receiving electrical energy from outside the couplant coating mechanism.

13. An ultrasonic imaging system, characterized by comprising:
a scanning assembly, comprising a scanning probe; and
the couplant coating mechanism comprising:
an engagement portion engaging with a scanning probe, the engagement portion being provided with a first channel; and
an accommodation portion connected to the engagement portion, the accommodation portion comprising a main body portion for accommodating a couplant, an internal space of the main body portion being in communication with the first channel, and the couplant accommodated in the main body portion flowing out along the first channel.

14. The ultrasonic imaging system according to claim 13, wherein
the scanning assembly further comprises a frame, the frame accommodating the scanning probe and a driving device, the driving device driving the scanning probe to move within the frame to perform ultrasonic scanning.

15. The couplant coating mechanism according to claim 13, wherein
the accommodation portion further comprises an extension portion connected to the main body portion, the extension portion is provided with a second channel that is in communication with the internal space of the main body portion, and the internal space of the main body portion is in communication with the first channel via the second channel.

16. The couplant coating mechanism according to claim 15, wherein
there are two extension portions oppositely disposed, and each extension portion is provided with the second channel.

17. The couplant coating mechanism according to claim 13, wherein
the engagement portion comprises two wall portions, and a connection portion connecting the two wall portions; and
a gap between the two wall portions is used for accommodating the scanning probe, and each wall portion is provided with the first channel.

18. The couplant coating mechanism according to claim 17, wherein
there are a plurality of first channels disposed extending from top to bottom.

19. The couplant coating mechanism according to claim 17, wherein
extending ends of the two wall portions each have a couplant outlet in communication with the first channel.

20. The couplant coating mechanism according to claim 19, wherein
the couplant outlet is an integral structure formed along the extending end, and the curvature of the couplant outlet matches that of an extending end of the scanning probe.
